(19) United States
(12) Patent Application Publication
(10) Pub. No.: US 2018/0085091 A1
(43) Pub. Date: Mar. 29, 2018

(54) ULTRASONIC MEASUREMENT DEVICE, AND METHOD OF CONTROLLING ULTRASONIC MEASUREMENT DEVICE

(71) Applicant: Seiko Epson Corporation, Tokyo (JP)

(72) Inventor: Masaki HAYASHI, Matsumoto (JP)

(21) Appl. No.: 15/709,922

(22) Filed: Sep. 20, 2017

(30) Foreign Application Priority Data
Sep. 26, 2016 (JP) 2016-187405

(51) Int. Cl. A61B 8/00; G01S 7/52

(52) U.S. Cl. CPC A61B 8/4494; G01S 7/52023; A61B 8/4427; A61B 8/56

(57) ABSTRACT

An ultrasonic measurement device includes an ultrasonic probe having a plurality of ultrasonic elements arranged, each of the ultrasonic elements being adapted to transmit and receive an ultrasonic beam, and an arithmetic processor adapted to perform a reduction process of reducing an amount of information of received signals received by the respective ultrasonic elements based on a reception frequency, then perform a beam forming process on the signals, on which the reduction process has been performed, to generate an ultrasonic image.

### BACKGROUND

### 1. Technical Field

The present invention relates to an ultrasonic measurement device and so on for performing ultrasonic measurement.

### 2. Related Art

In the past, there has been known an ultrasonic measurement device for performing a scan with an ultrasonic beam using an ultrasonic probe having a plurality of ultrasonic elements (ultrasonic vibrators) arranged, to thereby image an internal appearance of a living body. For performing imaging, there is performed a beam forming (BF) process for adding received signals received by the respective ultrasonic elements to each other. Since sufficient resolution of the image cannot be obtained by a simple beam forming process in some cases, technologies for obtaining an image with higher resolution are under development. For example, an adaptive beam forming process described in JP-A-2015-77393 (Document 1) is one of such technologies.

Incidentally, the adaptive beam forming process can provide higher resolution compared to the conventional beam forming process of a non-adaptive type on the one hand, but causes a problem of increasing an amount of calculation on the other hand. As a technology for solving the problem, there can be cited, for example, a technology in JP-A-2011-5237 (Document 2). The technology of Document 2 is for achieving speeding-up of the signal processing by adding echo detection data (received signals) from the channels adjacent to each other to thereby thin the data, and then performing the adaptive signal processing (the adaptive beam forming process).

According to the technology of Document 2, since it is possible to reduce the number of received signals as the processing target of the adaptive beam forming process, the amount of calculation can be reduced accordingly. However, if the received signals from the channels adjacent to each other are simply added to each other, the advantage of the adaptive beam forming process for improving the resolution is attenuated, and thus the image quality of the ultrasonic image generated is affected in some cases. Further, it is useful if the amount of calculation can also be reduced without damaging the image quality in the case of performing the non-adaptive beam forming process.

### SUMMARY

An advantage of some aspects of the invention is to reduce an amount of calculation related to the execution of the beam forming process while suppressing the deterioration of the image quality of the ultrasonic image.

A first aspect of the invention is directed to an ultrasonic measurement device including an ultrasonic probe having a plurality of ultrasonic elements arranged, each of the ultrasonic elements being adapted to transmit and receive an ultrasonic beam, and an arithmetic processor adapted to perform a reduction process of reducing an amount of information of received signals received by the respective ultrasonic elements based on a reception frequency, then perform a beam forming process on the signals, on which the reduction process has been performed, to generate an ultrasonic image.

As another aspect of the invention, the invention may be configured as a method of controlling an ultrasonic measurement device adapted to perform ultrasonic measurement using an ultrasonic probe having a plurality of ultrasonic elements arranged, each of the ultrasonic elements being adapted to transmit and receive an ultrasonic beam, the method including the steps of performing a reduction process of reducing an amount of information of received signals received by the respective ultrasonic elements based on a reception frequency, and generating an ultrasonic image by performing a beam forming process on the signals on which the reduction process has been performed.

According to the first aspect or the like of the invention, prior to the beam forming process, the amount of the information of the received signals received by the respective ultrasonic elements can be reduced based on the reception frequency. According to this aspect of the invention, it becomes possible to reduce the amount of the calculation related to the execution of the beam forming process while suppressing the deterioration of the image quality of the ultrasonic image.

As a second aspect of the invention, the ultrasonic measurement device according to the first aspect of the invention may be configured such that the arithmetic processor performs, as a part of the reduction process, a frequency analysis process of performing frequency analysis on the received signals of the respective ultrasonic elements to transform the received signals into a plurality of frequency signals, and a selection process of selecting signals with given frequency components from the frequency signals to thereby eliminate a signal other than the given frequency components.

According to the second aspect of the invention, it is possible to possible to perform the beam forming process in which the signals with the given frequency components are selected from the plurality of frequency signals obtained by performing frequency analysis on the received signals corresponding respectively to the ultrasonic elements, and then the signals with the given frequency components are used. By eliminating the frequency signals having a small influence on the image quality of the ultrasonic image, it becomes possible to reduce the amount of the calculation related to the execution of the beam forming process while suppressing the deterioration of the image quality of the ultrasonic image.

As a third aspect of the invention, the ultrasonic measurement device according to the second aspect of the invention may be configured such that the arithmetic processor performs setting an allowable arrival angle range of a side lobe allowed to be received, obtaining a selection proportion of the ultrasonic elements corresponding to the allowable arrival angle range, and the selection process using components corresponding to the selection proportion on a low frequency side out of the reception frequencies obtained by the frequency analysis as the given frequency components.

According to the third aspect of the invention, it is possible to set the allowable arrival angle range of the side lobe allowed to be received to obtain the corresponding selection proportion. Further, it is possible to perform the beam forming process in which the signals of the frequency components on the low frequency side corresponding to the selection proportion are selected from the plurality of frequency signals, and the signals of the frequency components on the low frequency side corresponding to the selection proportion are used.

As a fourth aspect of the invention, the ultrasonic measurement device according to the third aspect of the invention may be configured such that the arithmetic processor sets an allowable level of the side lobe allowed to be received to thereby set the allowable arrival angle range fulfilling the allowable level based on reception directional characteristics related to the ultrasonic probe.

According to the fourth aspect of the invention, it is possible to set the allowable level of the side lobe allowed to be received. Further, by setting the allowable level, the angular range fulfilling the allowable level in the reception directional characteristics related to the ultrasonic probe can be set as the allowable arrival angle range.

As a fifth aspect of the invention, the ultrasonic measurement device according to the third aspect of the invention may be configured such that the arithmetic processor sets the allowable arrival angle range in accordance with depth of a processing target point of the beam forming process.

According to the fifth aspect of the invention, the allowable arrival angle range can be set in accordance with the depth of the processing target point for each of the processing target points of the beam forming process.

As a sixth aspect of the invention, the ultrasonic measurement device according to any one of the first through fifth aspects of the invention may be configured such that the arithmetic processor performs, as a part of the reduction process, a thinning process adapted to thin the received signals corresponding respectively to the ultrasonic elements in accordance with depth of a processing target point of the beam forming process.

According to the sixth aspect of the invention, the received signals can be thinned in accordance with the depth of the processing target point for each of the processing target points of the beam forming process. Further, it is possible to perform the beam forming process with respect to the received signals on which the thinning process has been performed.

As a seventh aspect of the invention, the ultrasonic measurement device according to the sixth aspect of the invention may be configured such that the arithmetic processor performs the thinning process by thinning the received signals based on a pitch length of the ultrasonic elements corresponding to a “propagatable” frequency determined based on depth of the processing target point.

According to the seventh aspect of the invention, it is possible to thin the received signals based on the pitch length of the ultrasonic elements corresponding to the “propagatable” frequency determined by the depth of the processing target point.

As an eighth aspect of the invention, the ultrasonic measurement device according to any one of the first through seventh aspects of the invention may be configured such that the arithmetic processor calculates weight based on the signals on which the reduction process has been performed, and performs the beam forming process as an adaptive beam forming process in which weighted addition is performed on the signals using the weight.

According to the eighth aspect of the invention, by performing the adaptive beam forming process, since the resolution (azimuth resolution) can be improved compared to the non-adaptive beam forming process, the image quality of the ultrasonic image can be improved.

### BRIEF DESCRIPTION OF THE DRAWINGS

The invention will be described with reference to the accompanying drawings, wherein like numbers reference like elements.

FIG. 1 is a diagram showing a system configuration example of an ultrasonic measurement device.

FIG. 2 is a diagram showing a processing block example of a reduction process.

FIG. 3 is a diagram showing a data configuration example of a number-of-reception channels table.

FIG. 4 is a diagram showing an example of reception directional characteristics.

FIG. 5 is a diagram showing a relationship between an incoming wave coming to ultrasonic elements and the arrival angle of the incoming wave.

FIG. 6 is a diagram showing the relationship between the incoming wave coming to ultrasonic elements and the arrival angle of the incoming wave.

FIG. 7 is a diagram showing the relationship between the incoming wave coming to ultrasonic elements and the arrival angle of the incoming wave.

FIG. 8 is a diagram showing a selection proportion conversion formula as a graph.

FIG. 9 is a block diagram showing a functional configuration example of the ultrasonic measurement device.

FIG. 10 is a flowchart showing a flow of a generation process of an ultrasonic image.

FIG. 11 is a diagram showing a relationship between an angular range of a transmission beam width and sensitivity.

FIG. 12 is a diagram showing another relationship between the angular range of the transmission beam width and the sensitivity.

FIG. 13 is a block diagram showing a functional configuration example of an ultrasonic measurement device according to a modified example.

FIG. 14 is a flowchart showing a flow of a generation process of an ultrasonic image in the modified example.

### DESCRIPTION OF AN EXEMPLARY EMBODIMENT

A preferred embodiment of the invention will hereinafter be described with reference to the accompanying drawings. It should be noted that the invention is not limited by the embodiment hereinafter described, and configurations to which the invention can be applied are not limited to the following embodiment. Further, in the description of the drawings, the same parts are denoted by the same symbols.

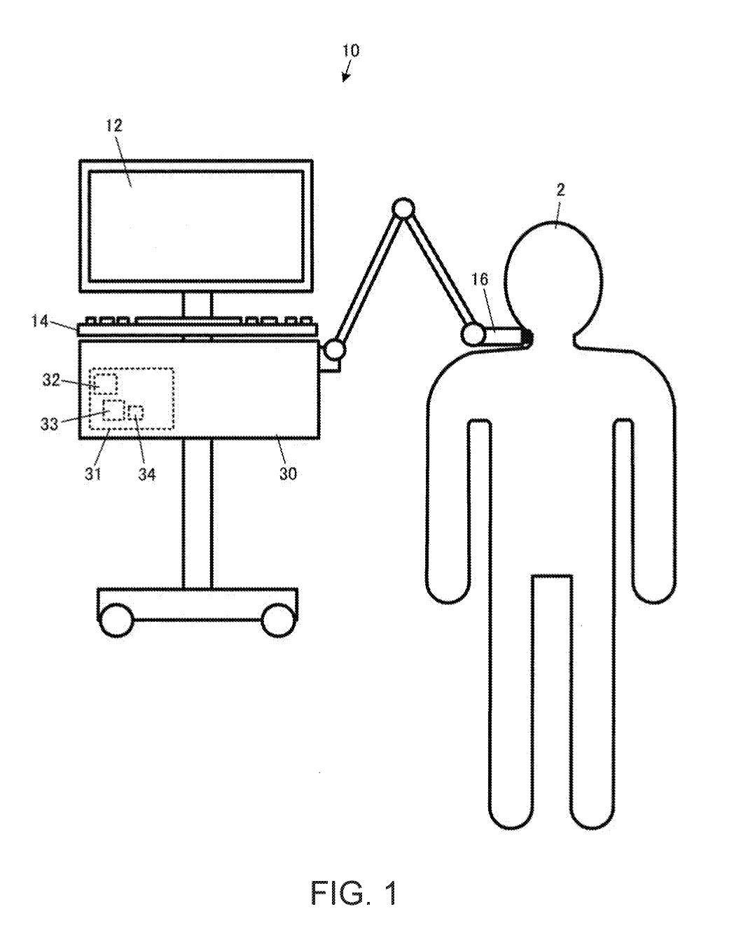

FIG. 1 is a diagram showing a system configuration example of an ultrasonic measurement device 10 according to the present embodiment. The ultrasonic measurement device 10 is for obtaining biological information of a test subject 2 using ultrasonic measurement, and is provided with a touch panel 12 functioning as both of a device for displaying an image of a measurement result and operational information and a device for inputting an operation, a keyboard 14 for inputting an operation, an ultrasonic probe (a probe) 16, and an image processing device 30.

The ultrasonic probe 16 incorporates a plurality of ultrasonic elements (ultrasonic vibrators) arranged in an array with regular intervals on a sensor surface, and performs the ultrasonic measurement with, for example, a so-called linear scanning method of transmitting and receiving an ultrasonic beam along a plurality of scan lines parallel to each other while shifting the incident position of the ultrasonic beam in the arrangement direction of the ultrasonic elements. The ultrasonic probe 16 is used with the sensor surface appressed against a biological surface (a cervical region in FIG. 1) of the test subject 2. It should be noted that the scan method is not limited to the linear scan method, and it is also possible to apply the present embodiment in a similar manner in the case of adopting other scan methods such as a sector scan method. Further, the measurement region against which the ultrasonic probe 16 is appressed is not limited to the cervical region, but is set to the region of the test subject 2 corresponding to the purpose of the measurement such as a wrist, an arm, or an abdominal region.

The image processing device 30 is equipped with a control board 31, and is connected to each part of the device such as the touch panel 12, the keyboard 14, or the ultrasonic probe 16 so as to be able to transmit and receive signals. On the control board 31, there are mounted a storage medium 33 such as an IC memory or a hard disc drive, and a communication IC 34 for realizing data communication with an external device besides a variety of integrated circuits such as a central processing unit (CPU) 32, an application specific integrated circuit (ASIC), and a field-programmable gate array (FPGA). The CPU 32 or the like executes a program stored in the storage medium 33 in the image processing device 30, and thus, the ultrasonic measurement device 10 performs a process necessary for obtaining the biological information such as the ultrasonic measurement.

Specifically, due to the control by the image processing device 30, the ultrasonic measurement device 10 transmits the ultrasonic beam from the ultrasonic probe 16 to the test subject 2, and then receives the reflected wave of the ultrasonic beam to perform the ultrasonic measurement. Then, the ultrasonic measurement device 10 amplifies/performs signal processing on the received signal of the reflected wave to generate reflected wave data such as positional information of an intravital structure of the test subject 2 or a temporal change. The ultrasonic measurement is repeatedly performed with a predetermined period. A measurement unit is called “frame.”

The reflected wave data includes at least a so-called B-mode image, but it is also possible to assume that the reflected wave data includes images of so-called A-mode, M-mode, and color Doppler mode besides the B-mode image. The A-mode is the mode for displaying the amplitude (an A-mode image) of the reflected wave defining the first axis as the sampling point sequence of the received signal along the transmission/reception direction (the direction of the scan line), and the second axis as the received signal intensity of the reflected wave at each of the sampling points. Further, the B-mode is the mode for displaying a two-dimensional ultrasonic image (a B-mode image) of the intravital structure which is visualized by converting the reflected wave amplitude (the A-mode image) obtained while scanning a predetermined scan range with the ultrasonic beam into a luminance value.

### Principle

When generating the reflected wave data, the ultrasonic measurement device 10 performs (received beam forming) a process of performing phasing addition on the received signals from the respective ultrasonic elements (hereinafter also referred to as “channels”) for each of the sampling points. In the case in which a plurality of ultrasonic element groups constitutes one channel to perform transmission and reception of the ultrasonic wave, the phasing addition is performed on the received signals obtained by the respective ultrasonic element groups. Hereinafter, the received signal from each of the channels each constituted by the ultrasonic element or the ultrasonic element group is referred to as a “channel signal.”

Specifically, after a reception focusing process (a phasing process) for applying a delay to the channel signal from each of the channels, the beam forming process for adding the channel signals, on which the reception focusing process has been performed, to each other is performed. Thus, it is possible to amplify only the signal from a desired direction (the direction of the scan line) having the same phase, and thus it is possible to extract the desired wave from the direction of the scan line.

Here, as one of the methods of the wave forming process, there is known adaptive beam forming (hereinafter referred to as an “adaptive BF process”) of dynamically changing the addition weight used for the addition of the channel signals in accordance with the incoming wave. According to a brief description of the processing procedure of the adaptive beam forming, the following process is performed for each of the sampling points. Firstly, a correlation matrix is calculated based on the channel signals of the respective channels on which the reception focusing process has been performed. Subsequently, the addition weight to be multiplied by each of the channel signals is calculated based on the correlation matrix thus calculated using a steering vector defined based on the direction of the scan line. Subsequently, the weighted addition is performed on the channel signals of the respective channels, on which the reception focusing process has been performed, using the addition weights thus calculated. As a specific example of the adaptive BF process, there can be cited a minimum variance (MV) method, an amplitude and phase estimation (APES) method and so on, which can arbitrarily be adopted. According to the adaptive BF process, it is possible to perform the weighted addition on the channel signals with a restriction on the direction so as to have sensitivity only to the desired wave from the direction of the scan line and so as not to have sensitivity to unwanted waves, and thus high resolution can be achieved.

However, since the adaptive BF process is a complicated process of calculating the addition weights to be multiplied by the channel signals every time, there is a problem that an amount of calculation increases. Here, the amount of calculation related to the execution of the adaptive BF process is determined by the number M of the channels and the degree of the calculating formula for calculating the addition weight, and is expressed by the O notation as O(M̂3). Therefore, if the number of the signals passed to the adaptive BF process can be made smaller than the number M of the channels, an amount of data processed in the adaptive BF process can be reduced, and thus, the amount of calculation can be reduced.

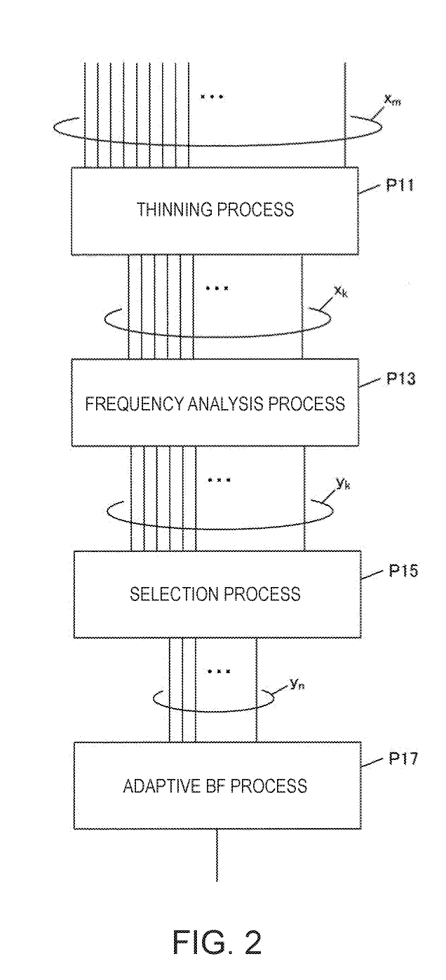

Therefore, in the present embodiment, prior to the adaptive BF process, there is performed a reduction process for reducing an amount of information of the channel signals from the respective channels based on the reception frequency. FIG. 2 is a diagram showing a processing block example of the reduction process. In the reduction process, firstly, (1) a thinning process P11 is performed on the channel signals (in more detail, the channel signals on which the focusing process has been performed) xₘ from the respective channels, the number of which is M. Subsequently, (2) there is performed a frequency analysis process P13 of the channel signals xₖ the number of which is reduced to K (M≧K) by the thinning process P11, and then there is performed a selection process P15 for selecting signals with given frequency components from the frequency signals yₖ obtained by the frequency analysis process. The frequency signals yₙ the number of which is reduced to N (K≧N) by the selection process P15 is passed to the adaptive BF process P17.

### (1) Thinning Process

The ultrasonic wave having entered the test subject 2 propagates through the test subject 2 while being attenuated. Therefore, the frequency (“propagatable” frequency) of the carrier wave which can propagate to the sampling point (processing target point) regarded as the processing target by the adaptive BF process P17 differs by the depth of the processing target point from the biological surface.

Here, the interval between (pitch length of) the ultrasonic elements (channels) provided to the probe 16 is set to the length corresponding to a half wavelength of the carrier wave according to the sampling theorem. Therefore, in the case in which the pitch length of the ultrasonic elements is determined in accordance with the maximum carrier wave frequency, assuming that the actual carrier wave frequency decreases by half in the process of propagating through the test subject 2, the wavelength of the carrier wave is doubled, and therefore, it results that the pitch length twice of the original pitch length is only required as the necessary pitch length. In terms of the number of channels, half the number is sufficient. Therefore, in the thinning process P11, the channel signals xₘ of the respective channels are thinned in accordance with the depth of the processing target point, and then the channel signals xₖ as a result of the thinning process P11 is passed to the frequency analysis process P13.

In order to do this, the relationship between the frequency (reception frequency) of the incoming wave assumed from the “propagatable” frequency of the depth and the necessary number of channels (number of the reception channels) is determined in advance for each depth to form a number-of-reception channels table. Specifically, the reception frequency is calculated and set using a simplified model of the attenuation and taking the attenuation of the ultrasonic wave corresponding to the depth into consideration. Alternatively, it is also possible to set the reception frequency by measuring the reception frequency for each depth. Meanwhile, the number of the reception channels is set by identifying the necessary pitch length for each of the reception frequencies of the respective depths thus set in accordance with the relationship between the carrier wave frequency and the pitch length described above.

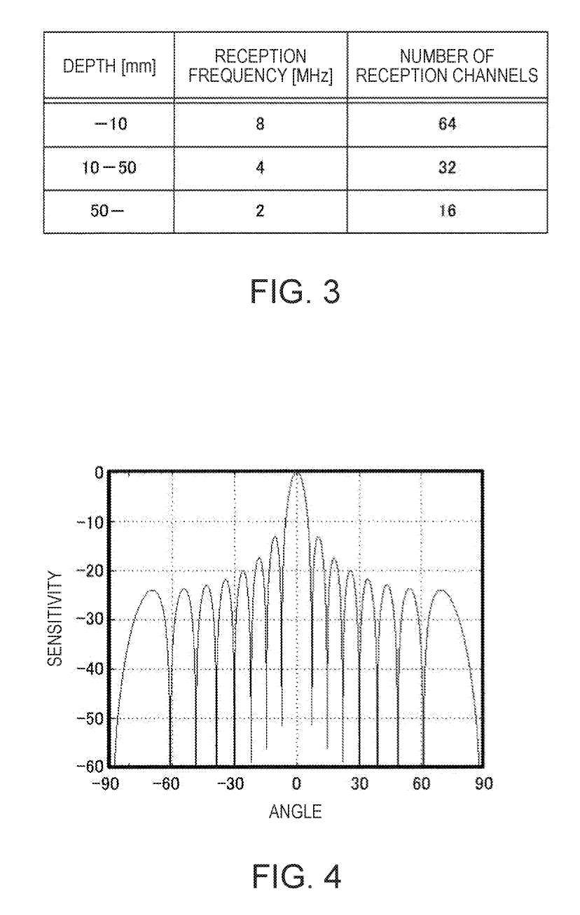

FIG. 3 is a diagram showing a data configuration example of the number-of-reception channels table. As shown in FIG. 3, the correspondence relationship between the depth, the reception frequency, and the number of the reception channels is set in the number-of-reception channels table. In the setting example shown in FIG. 3, the reception frequency corresponding to the depth smaller than 10 [mm] is 8 [MHz] on the one hand, the reception frequency decreases by half to 4 [MHz] in the depth no smaller than 10 [mm] and no larger than 50 [mm]. Therefore, to the number of the reception channels in the case of the depth no smaller than 10 [mm] and no larger than 50 [mm], “32” is set, which is a half of the number of the reception channels of “64” in the case of the depth smaller than 10 [mm]. Further, in the depth larger than 50 [mm], the reception frequency further decreases by half to 2 [MHz], and therefore, “16” is set to the number of the reception channels.

Here, in the description of the thinning process P11 on the assumption that the setting of the aperture width of the ultrasonic elements (channels) used for each scan is 64 channels, with respect to the processing target points with the depth smaller than 10 [mm], the number of the reception channels is “64,” and therefore, the channel signals xₘ from the respective channels are directly passed to the frequency analysis process P13 in the posterior stage as the channel signals xₖ (M=K) without thinning the channel signals xₘ. In contrast, in the case in which the depth of the processing target point is no smaller than 10 [mm] and no larger than 50 [mm], the number of the reception channels is “32” half as large as the number of the total channels of 64, and therefore, the channel signals xₘ are thinned alternately so that the necessary pitch length becomes twice as large as the actual pitch length. Then, the 32 channel signals xₖ obtained by the thinning operation are passed to the frequency analysis process P13. Further, in the case in which the depth exceeds 50 [mm], since the number of the reception channels is “16” quarter as large as the number of the total channels of 64, the channel signals xₘ are thinned by eliminating 3 signals every 4 signals to obtain 16 channel signals xₖ so that the necessary pitch length becomes four times, and then the 16 channel signals xₖ are passed to the frequency analysis process P13.

### (2) Frequency Analysis Process/Selection Process

FIG. 4 is a diagram showing an example of reception directional characteristics (a directionality pattern) of the incoming wave from a variety of directions when providing the directionality to 0 degree defining the horizontal axis as an angle (arrival angle) and the vertical axis as sensitivity (reception sensitivity). The reception directional characteristics can be obtained by the following formula (1) using the carrier frequency and the aperture width. The aperture width is determined based on the number M of the channels used and the pitch length, and is designated by the positions dₘ of the respective M ultrasonic elements in the formula (1). Further, in the formula (1), “c” represents the sound speed, “f” represents the carrier wave frequency, “6” represents the arrival angle, and “wₖ” represents the weights of the respective channels. The reception directional characteristics shown in FIG. 4 are obtained assuming that the number M of the channels is “16,” the pitch length is a half wavelength of the carrier wave, and the weight wₖ is “1.”

$${E}_{sum} ( θ ) = {\sum }_{m = 1}^{M} {w}_{k} exp ( - j 2 π f \frac{{d}_{m}}{c} sin θ ) \qquad ( 1 )$$

As shown in FIG. 4, in the reception directional characteristics, the main lobe appears in the direction of 0 degree in which the directionality is provided, and the side lobes appear in the directions deviated from 0 degree. In short, the main lobe is the desired wave, and the side lobes are unwanted waves. Therefore, the side lobes high in sensitivity degrade the resolution, and incur the deterioration of the image quality of the ultrasonic image.

However, this does not necessarily arise the problem in the entire angular range other than 0 degree. Since the longer the distance of the side lobe from 0 degree is, the lower the level of the side lobe becomes, at the angle at which the level of the side lobe is as low as a negligible level, even if the wave comes from that direction, the wave does not become a factor for dramatically degrading the resolution. In addition, since the ultrasonic beam is transmitted toward the focal position on the scan line with the beam converged, in general, the closer to the direction (direction of 0 degree) of the scan line, the higher the intensity of the received signal becomes, and the further from 0 degree, the lower the intensity of the received signal becomes. Therefore, even if the adaptive BF process P17 is performed while neglecting the waves with large arrival angle, the influence on the image quality is small. Further, by eliminating the signal components related to the negligible incoming wave from the channel signals (the channel signals xₖ after the thinning process in the present embodiment) of the respective channels to thereby reduce the number of the signals to be passed to the adaptive BF process P17, the amount of the calculation related to the execution of the adaptive BF process P17 can be reduced accordingly.

For example, assuming that the allowable level (hereinafter referred to as an “allowable sensitivity level”) is set to −20 [dB], in the example shown in FIG. 4, it is possible to neglect the incoming waves the arrival angle of which is within the angular range (an allowable arrival angle range) of equal to or greater than about ±30 degrees. Therefore, due to the frequency analysis process P13 and the selection process P15, the signal components related to the incoming waves within the allowable arrival angle range are eliminated. The allowable sensitivity level is set by, for example, receiving the operation input by the user. It should be noted that it is also possible to adopt a configuration of setting the allowable sensitivity level in advance as a predetermined value (e.g., −20 [dB]).

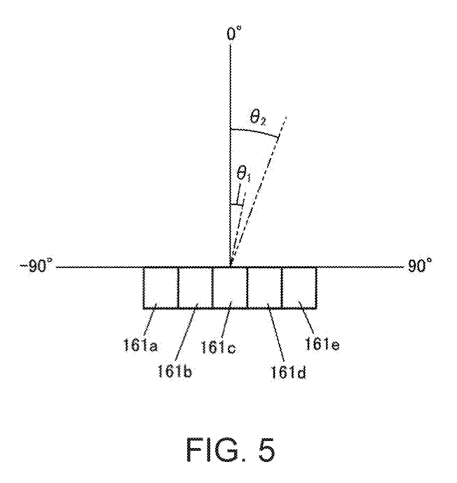

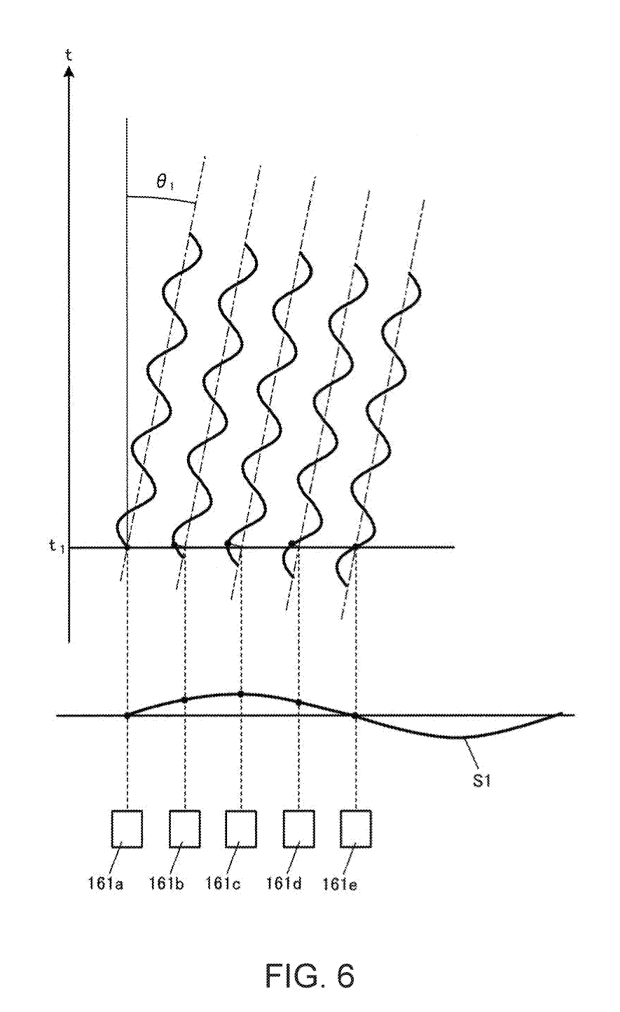

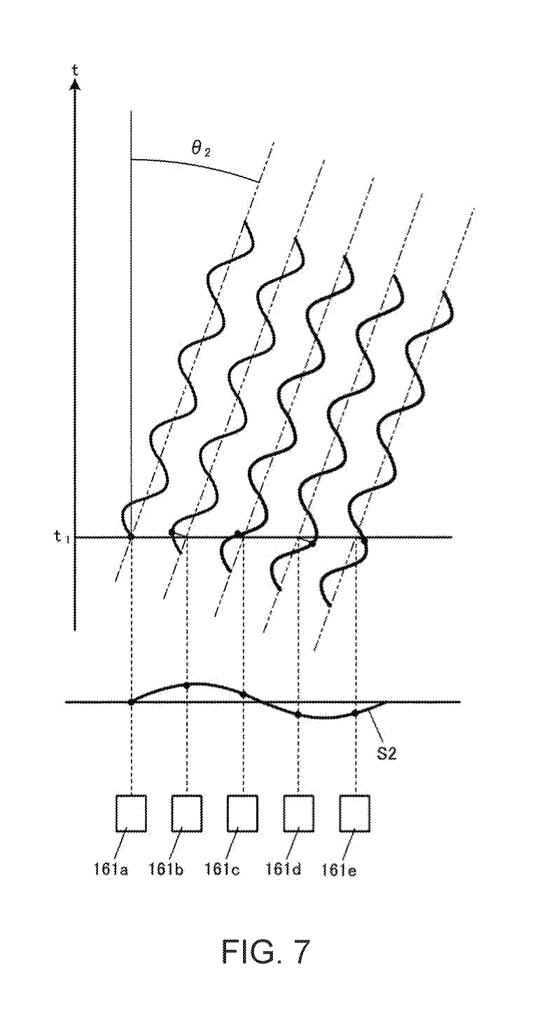

Incidentally, there is a predetermined relationship between the received wave (the incoming wave) coming to the ultrasonic element used and the arrival angle at which the incoming wave comes. Hereinafter, the relationship described above will be described with reference to FIGS. 5 to 7 and citing the ideal state of the case, in which the incoming wave is a single wave with the carrier wave frequency, and comes to the ultrasonic elements as a parallel wave, as an example. FIG. 5 is a schematic diagram showing the arrival angle θ of the incoming wave coming to the ultrasonic elements 161a through 161e. It should be noted that in FIG. 5, the number of the channels used is set to “5” for the sake of simplification, and the five ultrasonic elements 161a through 161e are shown. Further, FIG. 6 is a schematic diagram showing the reception of the incoming wave from the arrival angle θ₁ shown in FIG. 5, and FIG. 7 is a schematic diagram showing the reception of the incoming wave from the arrival angle θ₂ shown in FIG. 5.

For example, in the case in which the arrival angle is 0 degree, the phases of the incoming waves received by the respective ultrasonic elements 161a through 161e are the same. Therefore, in the case of performing an inclusive frequency analysis (hereinafter simply referred to as a “frequency analysis”) with the uniform reception timing on the received signals (the channel signals) of the respective ultrasonic elements 161a through 161e, the signal level of the signal (0 [Hz]) corresponding to a direct-current signal becomes the highest out of the frequency signals.

In contrast, in the case in which the arrival angle is an angle other than 0 degree (e.g., θ₁ or θ₂) such as 15 degrees or 30 degrees, differences occur between the phases of the incoming waves received by the respective ultrasonic elements 161a through 161e as illustrated in FIG. 6 and FIG. 7. Due to the phase differences, phase difference signals are generated between the received signals of the respective ultrasonic elements 161a through 161e. In other words, due to the fact that the arrival angle is not equal to 0 degree, differences are caused between the signal levels of the incoming waves received by the respective ultrasonic elements 161a through 161e, and if the signal levels are observed along the arrangement of the ultrasonic elements 161a through 161e, a cyclic signal corresponding to the arrival angle is obtained. This signal is referred to as a “phase difference signal.” In the middle of FIG. 6, there is shown an example of the phase difference signal S1 at the time point t₁ in the case in which the arrival angle is θ₁, and in the middle of FIG. 7, there is shown an example of the phase difference signal S2 at the time point t₁ in the case in which the arrival angle is θ₂. As represented by the waveforms of the respective phase difference signals S1, S2, as the arrival angle approaches 90 degrees, the cycle of the phase difference signal is shortened (the frequency rises). Then, when the arrival angle reaches 90 degrees, the frequency of the phase difference signal becomes equal to the frequency of the incoming wave, namely the carrier wave frequency.

Therefore, if the frequency analysis is performed on the received signals of the respective ultrasonic elements 161a through 161e in the case in which the arrival angle is not equal to 0 degree, the level of the signal with a certain frequency becomes the highest. If the frequency of the signal with the highest signal level is equal to the frequency (=the carrier wave frequency) of the incoming wave, it is possible to determine that the arrival angle is 90 degrees.

The above is the case of the ideal state of the case in which the incoming wave is the single wave with the carrier wave frequency and comes to the ultrasonic elements as the parallel wave, but can also be applied to the actual received signals. Specifically, when performing the frequency analysis on the received signals of the respective ultrasonic elements 161a through 161e, it results that a plurality of frequency signals is detected between 0 [Hz] through the carrier wave frequency. Further, the range of 0 [Hz] through the carrier wave frequency corresponds to the arrival angle of 0 degree through 90 degrees (more accurately, ±90 degrees). Therefore, the elimination of the signal component related to the incoming wave in the allowable arrival angle range described above can be achieved by eliminating the frequency signal in the frequency band on the high frequency side corresponding to the allowable arrival angle range, in other words, by selecting the frequency signal in the frequency band on the low frequency side not corresponding to the allowable arrival angle range.

Specifically, it is possible to perform the process of, for example, performing the discrete Fourier transform (DFT) due to the following formulas (2), (3) on the K channel signals xₖ using a beam space method as a known technology to thereby transform the K channel signals xₖ into the K frequency signals yₖ as the frequency analysis process P13.

$$\begin{matrix}{y}_{p + 1} = {\sum }_{j = 0}^{M - 1} {ω}^{jp} {x}_{j + 1} & ( 2 ) \\ where , ω = {e}^{- 2 π i / M} & ( 3 )\end{matrix}$$

Further, the selection process P15 is a process of selecting the frequency signals yₖ on the low frequency side from the frequency signals yₖ obtained by the frequency analysis to eliminate the frequency signals yₖ on the high frequency side. The number of the signals selected is determined using the relational expression (a selection proportion conversion formula) between the allowable arrival angle range determined in advance and the selection proportion. Specifically, the selection proportion is obtained from the allowable arrival angle range in accordance with the selection proportion conversion formula, and then the selection proportion thus obtained is multiplied by the number K (the number of the channel signals xₖ) of the frequency signals yₖ to obtain the number of the signals selected. Then, the frequency signals yₖ corresponding to the number of the signals selected are selected from the low frequency side out of the frequency signals yₖ to obtain the frequency signals yₙ, and then the frequency signals yₙ are passed to the adaptive BF process P17.

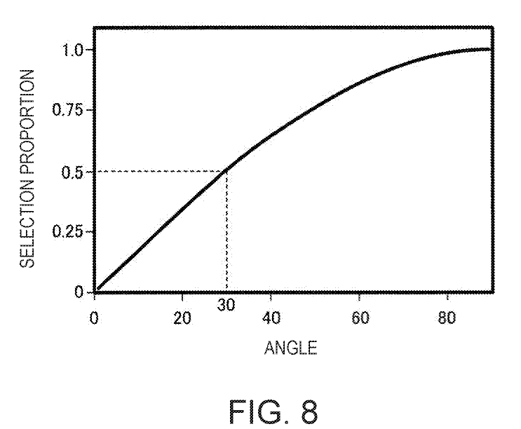

FIG. 8 is a diagram showing the selection proportion conversion formula as a graph. For example, in the case in which the allowable arrival angle range is equal to or larger than ±30 degrees, the number of the signals selected is determined using the selection proportion “0.5” corresponding to 30 degrees in the example shown in FIG. 8. In this case, it results that the K/2 frequency signals yₙ on the low frequency side are selected from the frequency signals yₖ, and it is possible to reduce the frequency signals yₖ by half and then pass the reduced frequency signals to the adaptive BF process P17. Therefore, it is possible to reduce the amount of the calculation related to the execution of the adaptive BF process P17 while suppressing the influence on the image quality.

### Functional Configuration

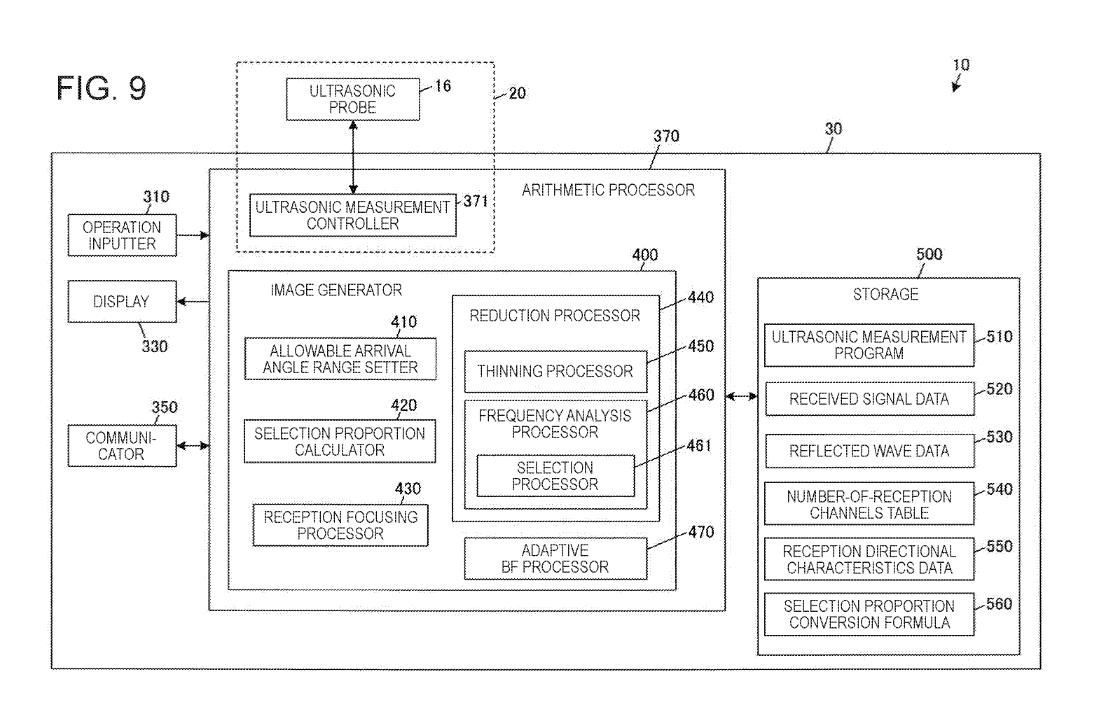

FIG. 9 is a block diagram showing a functional configuration example of the ultrasonic measurement device 10. The ultrasonic measurement device 10 is provided with an image processing device 30 and the ultrasonic probe 16, and the image processing device 30 is provided with an operation inputter 310, a display 330, a communicator 350, an arithmetic processor 370, and a storage 500.

The ultrasonic probe 16 is provided with the plurality of ultrasonic elements (channels) arranged, and transmits the ultrasonic wave based on a pulse voltage from the image processing device 30 (in more detail, an ultrasonic measurement controller 371 of the arithmetic processor 370). Then, the ultrasonic probe 16 receives the reflected wave of the ultrasonic wave thus transmitted, and then outputs the channel signals from the respective channels to the ultrasonic measurement controller 371.

The operation inputter 310 receives a variety of operations by the user, and outputs an operation input signal corresponding to the operation input to the arithmetic processor 370. The operation inputter 310 can be realized by a button switch, a lever switch, a dial switch, a track pad, a mouse, and so on. In FIG. 1, the touch panel 12 and the keyboard 14 correspond to the operation inputter 310.

The display 330 is realized by a display device such as a liquid crystal display (LCD), and performs a variety of types of display based on a display signal from the arithmetic processor 370. In FIG. 1 the touch panel 12 corresponds to the display 330.

The communicator 350 is a communication device for transmitting and receiving data with the outside under the control by the arithmetic processor 370. As the communication method of the communicator 350, it is possible to apply a variety of methods such as a type of achieving wired connection via a cable compliant with a predetermined communication standard, a type of achieving connection via an intermediate device also used as a battery charger called a cradle and so on, or a type of achieving wireless connection using wireless communication. In FIG. 1, the communication IC 34 corresponds to the communicator 350.

The arithmetic processor 370 is realized by a microprocessor such as a CPU or a graphics processing unit (GPU) and electronic components such as an ASIC, an FPGA, and an IC memory. Further, the arithmetic processor 370 performs the input/output control of the data between functional parts, and executes a variety of types of arithmetic processing based on a predetermined program and data, the operation input signal from the operation inputter 310, the channel signals of the respective channels from the ultrasonic probe 16, to thereby calculate the biological information of the test subject 2. In FIG. 1 the CPU 32 corresponds to the arithmetic processor 370. It should be noted that it is possible to assume that the parts constituting the arithmetic processor 370 are formed of hardware such as a dedicated modular circuitry.

The arithmetic processor 370 includes the ultrasonic measurement controller 371 and an image generator 400.

The ultrasonic measurement controller 371 constitutes an ultrasonic measurer 20 together with the ultrasonic probe 16, and the ultrasonic measurer 20 performs the ultrasonic measurement. The ultrasonic measurement controller 371 can be realized using a known technology. Specifically, the ultrasonic measurement controller 371 controls the transmission timing of the ultrasonic pulse by the ultrasonic probe 16, and generates the pulse voltage at the transmission timing, and then outputs the pulse voltage to the ultrasonic probe 16. On this occasion, the ultrasonic measurement controller 371 performs the transmission delay process to adjust the output timing of the pulse voltage to each of the channels. Further, the ultrasonic measurement controller 371 performs amplification and the filter process on the channel signals of the respective channels from the ultrasonic probe 16, and then outputs the channel signals (the measurement result) of the respective channels to the image generator 400.

The image generator 400 generates the ultrasonic image based on the channel signals of the respective channels from the ultrasonic measurement controller 371. The image generator 400 includes an allowable arrival angle range setter 410, a selection proportion calculator 420, a reception focusing processor 430, a reduction processor 440, and an adaptive BF processor 470.

The allowable arrival angle range setter 410 sets the allowable sensitivity level in accordance with the user operation, and sets the allowable arrival angle range using the allowable sensitivity level. The selection proportion calculator 420 calculates the selection proportion in accordance with the allowable arrival angle range set by the allowable arrival angle range setter 410.

The reception focusing processor 430 performs the reception focusing process of applying the delay to the channel signals of the respective channels by adding the delay time determined in advance for the corresponding channel. The channel signals xₘ of the respective channels on which the reception focusing process has been performed are output to a thinning processor 450 of the reduction processor 440.

The reduction processor 440 is provided with the thinning processor 450 and a frequency analysis processor 460, and performs the reduction process. The thinning processor 450 performs the thinning process for thinning the channel signals xₘ of the respective channels on which the focusing process has been performed in accordance with the depth of the processing target point. The channel signals xₖ on which the thinning process has been performed are output to the frequency analysis processor 460. The frequency analysis processor 460 performs the frequency analysis process for performing the frequency analysis on the channel signals xₖ to convert the channels signals xₖ into the plurality of frequency signals yₖ. The frequency analysis processor 460 is provided with a selection processor 461. The selection processor 461 performs the selection process for selecting the frequency signals yₙ on the low frequency side from the plurality of frequency signals yₖ obtained by the frequency analysis. The frequency signals yₙ on which the selection process has been performed are output to the adaptive BF processor 470.

The adaptive BF processor 470 performs the adaptive BF process on the frequency signals yₙ.

The storage 500 is realized by a storage medium such as an IC memory, a hard disc drive, or an optical disk. In the storage 500, there are stored a program for operating the ultrasonic measurement device 10 and realizing a variety of functions provided to the ultrasonic measurement device 10, and the data used during the execution of the program in advance, or temporarily in every processing. In FIG. 1, the storage medium 33 mounted on the control substrate 31 corresponds to the storage 500. It should be noted that the connection between the arithmetic processor 370 and the storage 500 is not limited to the connection with the internal bus circuit in the device, but can also be realized by a communication network such as a local area network (LAN) or the Internet. On that occasion, it is also possible to assume that the storage 500 is realized by an external storage device separate from the ultrasonic measurement device 10.

Further, the storage 500 stores an ultrasonic measurement program 510, received signal data 520, reflected wave data 530, the number-of-reception channels table 540, reception directional characteristics data 550, and the selection proportion conversion formula 560.

The arithmetic processor 370 retrieves and then executes the ultrasonic measurement program 510 to thereby realize the functions of the ultrasonic measurement controller 371, the image generator 400, and so on. It should be noted that in the case of realizing these functional parts with hardware such as an electronic circuit, the part of the program for realizing the functions can be eliminated.

As the received signal data 520, there are stored the received signals (the channel signals) of the respective ultrasonic elements (the channels) related to the scan of the scan line obtained as a result of the ultrasonic measurement.

As the reflected wave data 530, there is stored the reflected wave data obtained by the ultrasonic measurement repeated every frame. The reflected wave data 530 includes the data of the B-mode image for each frame as the ultrasonic image.

As illustrated in FIG. 3, the number-of-reception channels table 540 is a data table setting the correspondence relationship between the depth, the reception frequency, and the number of the reception channels.

As the reception directional characteristics data 550, there are stored the reception directional characteristics calculated using formula (1) (see FIG. 4). For example, in the case of fixing the aperture width used, the reception directional characteristics are calculated for each carrier wave frequency which can be selected, and the reception directional characteristics data 550 is generated for each carrier wave frequency in advance.

As the selection proportion conversion formula 560, there is stored the data of the selection proportion conversion formula as the relational expression between the allowable arrival angle range and the selection proportion shown in FIG. 8. It should be noted that besides the configuration of storing the selection proportion conversion formula, it is also possible to adopt a configuration of storing the relationship between the allowable arrival angle range and the selection proportion determined by the selection proportion conversion formula as a table.

### Flow of Process

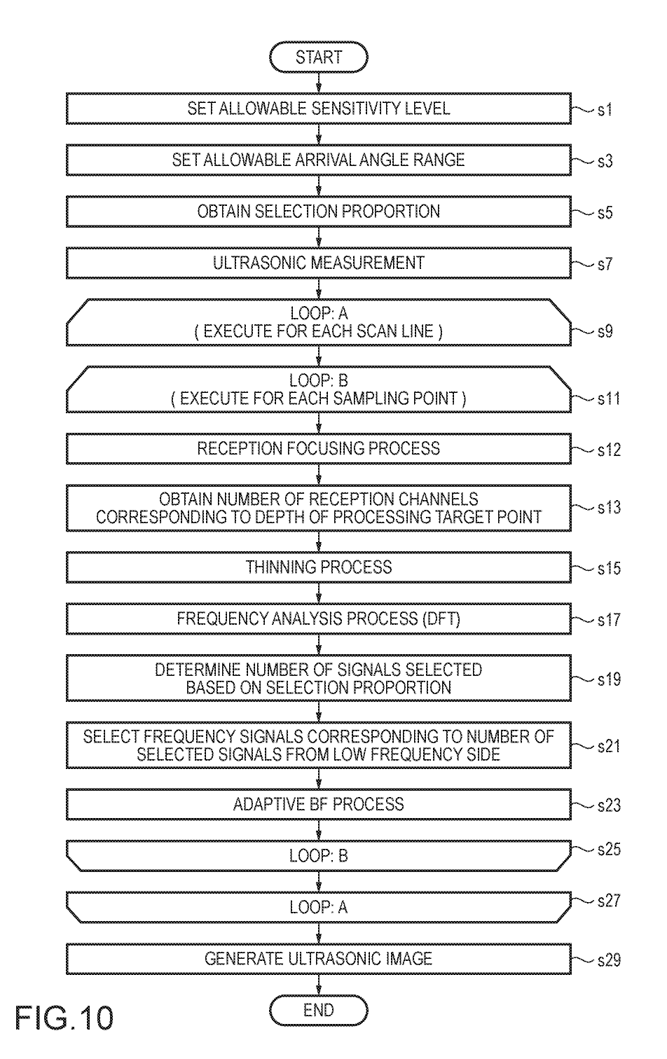

FIG. 10 is a flowchart showing a flow of a generation process of the ultrasonic image in the present embodiment. The process described here is started when, for example, the user presses the ultrasonic probe 16 against the body surface of the test subject 2, and then performs a predetermined measurement start operation. It should be noted that the present process can be realized by the arithmetic processor 370 retrieving the ultrasonic measurement program 510 from the storage 500 and then executing the ultrasonic measurement program 510 to thereby operate each section of the ultrasonic measurement device 10.

Prior to the ultrasonic measurement, firstly, the allowable arrival angle range setter 410 receives the operation input by the user to set (step s1) the allowable sensitivity level. Further, on this occasion, the selection operation of the carrier wave frequency is arbitrarily received. Then, the allowable arrival angle range setter 410 refers to the reception directional characteristics data 550 to read the angle corresponding to the allowable sensitivity level set in the step s1 from the reception directional characteristics of the carrier wave frequency, and then sets (step s3) the allowable arrival angle range.

Subsequently, the selection proportion calculator 420 obtains (step s5) the selection proportion corresponding to the angle obtained in the step s3 based on the selection proportion conversion formula 560. Subsequently, the process on and after the step S7 is repeated frame by frame.

Firstly, the ultrasonic measurer 20 performs (step s7) the ultrasonic measurement. Due to the process here, the measurement result is stored as the received signal data 520.

Subsequently, the process in loop A is repeated (step s9 through step s27) for each scan line while referring to the received signal data 520. Then, in the loop A, sampling for a certain period is performed with respect to the processing target line using the measurement result of the ultrasonic measurement in the step S7, and then the process in a loop B is performed (step s11 through step s25) sequentially setting the sampling points as the processing target point.

In the loop B, firstly, the reception focusing processor 430 performs (step s12) the reception focusing process of applying a delay of the delay time to the channel signals from the respective channels.

Subsequently, the thinning processor 450 retrieves the number of the reception channels corresponding to the depth of the processing target point from the number-of-reception channels table 540 to obtain (step s13) the number of the reception channels. Then, the thinning processor 450 thins (the thinning process; step s15) the channel signals xₘ of the respective channels on which the focusing process has been performed in accordance with the number of the reception channels obtained in the step s13.

Subsequently, the frequency analysis processor 460 performs the discrete Fourier transform (DFT) on the channel signals xₖ on which the thinning process has been performed using the beam space method to thereby transform (the frequency analysis process; step s17) the channel signals into the plurality of (K) frequency signals yₖ. Subsequently, the selection processor 461 determines (step s19) the number of the signals selected by multiplying the number K of the frequency signals obtained by the frequency analysis by the selection proportion obtained in the step s5. Then, the selection processor 461 selects (step s21) the frequency signals yₙ corresponding to the number of the signals selected from the low temperature side out of the frequency signals yₖ. Subsequently, the adaptive BF processor 470 performs (step s23) the adaptive BF process on the frequency signals yₙ on which the selection process has been performed.

The process in the loop B is repeated, and when the sampling on the processing target line has been completed, the process in the loop A with respect to the processing target line is terminated. Then, when the process in the loop A has been performed setting all of the scan lines as the processing target, the necessary process is performed on the output signal of the adaptive BF processor 470 obtained for each of the sampling points to generate (step s29) the ultrasonic image. The ultrasonic image thus generated is controlled to be displayed arbitrarily on the display 330 as the so-called B-mode image.

As described above, according to the present embodiment, it is possible to thin the channel signals xₘ from the respective channels in accordance with the depth of the processing target point of the adaptive BF process. Further, it is possible to reduce the number of channel signals by eliminating the signal components related to the incoming waves with a large arrival angle from the channel signals xₖ on which the thinning process has been preformed, and then perform the adaptive BF process. Therefore, it is possible to reduce the amount of the data processed in the adaptive BF to thereby reduce the amount of the calculation related to the execution of the beam forming process while suppressing the deterioration of the image quality of the ultrasonic image.

### Modified Example 1

In the embodiment described above, it is assumed that the allowable arrival angle range corresponding to the allowable sensitivity level is set in accordance with the reception sensitivity characteristics. In contrast, it is also possible to assume that the allowable arrival angle range is set in accordance with the depth of the processing target point.

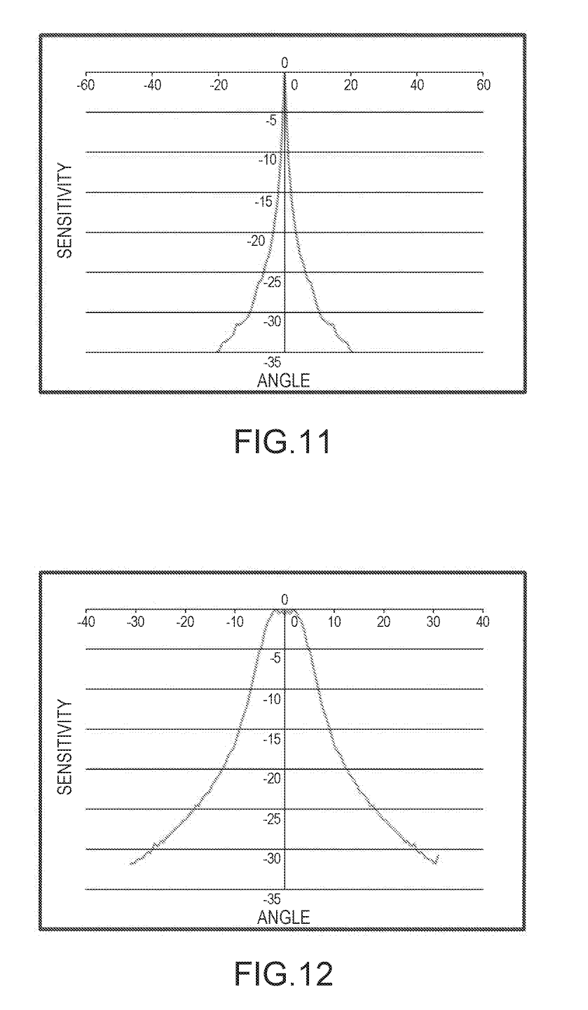

As described above, the ultrasonic beam transmitted from the ultrasonic probe 16 is a beam thinly converged toward the focal position. Therefore, from the viewpoint of receiving the incoming wave as the reflected wave, the narrower the beam width is, the less incoming light comes from the arrival angle deviated from 0 degree. The beam width (the transmission beam width) of the ultrasonic beam at each depth can be calculated from the shape of the ultrasonic beam transmitted and the aperture width. FIG. 11 is a diagram showing a relationship between the angular range of the transmission bean width and the sensitivity at the depth of 50 [mm] in the case in which the focus is 50 [mm], and FIG. 12 is a diagram showing the same relationship at the depth of 100 [mm].

Therefore, in the present modified example, the relationship between the angular range of the transmission beam width and the sensitivity is calculated in advance for each depth to generate transmission beam width data. Then, using the transmission beam width data corresponding to the depth of the processing target point, the allowable arrival angle range is set in accordance with the angular range of the transmission beam width corresponding to the allowable sensitivity level with which the reception is allowed.

For example, in the case in which the allowable sensitivity level is set to −20 [dB], and the depth of the processing target point is 50 [mm], the transmission beam width data defining the relationship of FIG. 11 is referred to. Then, the range equal to or larger than ±5 degrees which is out of the angular range of the transmission beam width at −20 [dB] is defined as the allowable arrival angle range. Further, in the case in which the depth of the processing target point is 100 [mm], the transmission beam width data defining the relationship of FIG. 12 is referred to. Then, the range equal to or larger than ±10 degrees which is out of the angular range of the transmission beam width at −20 [dB] is defined as the allowable arrival angle range. After setting the allowable arrival angle range, the selection proportion is obtained using the selection proportion conversion formula in substantially the same manner as in the embodiment described above.

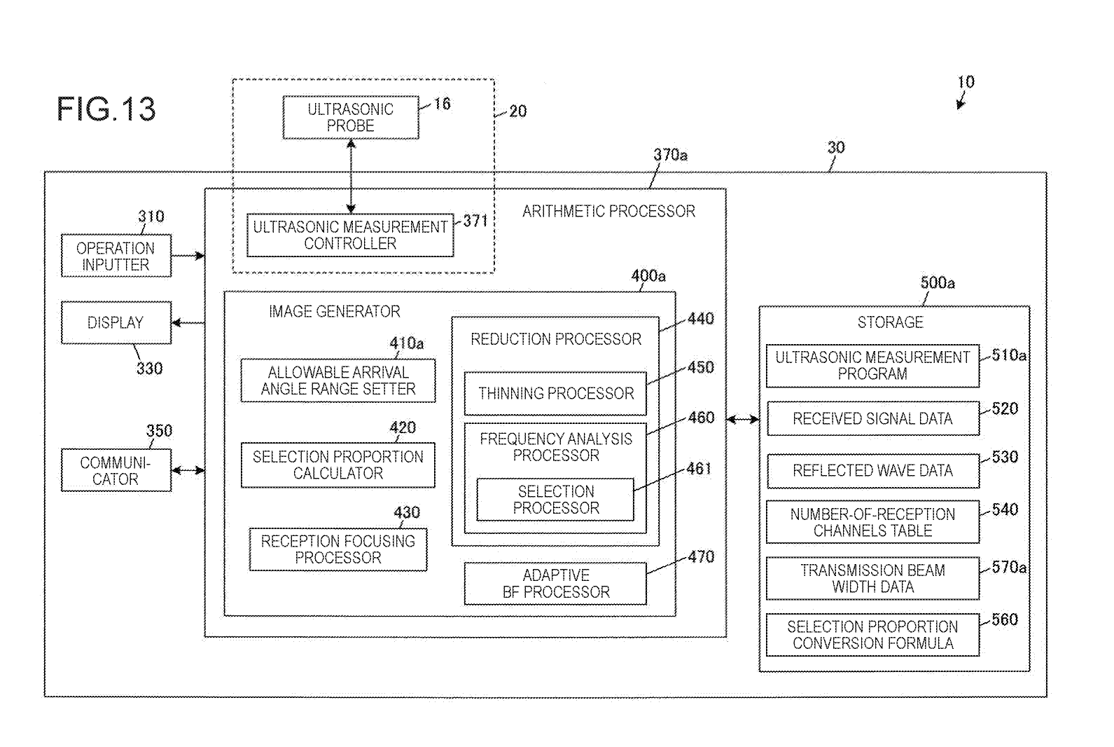

FIG. 13 is a block diagram showing a functional configuration example of the ultrasonic measurement device 10 according to the present modified example. It should be noted that in FIG. 13, substantially the same constituents as in the embodiment described above are denoted by the same symbols. In the ultrasonic measurement device 10 according to the present modified example, an image generator 400a of an arithmetic processor 370a includes an allowable arrival angle range setter 410a, the selection proportion calculator 420, the reception focusing processor 430, the reduction processor 440, and the adaptive BF processor 470. Further, a storage 500a stores an ultrasonic measurement program 510a, the received signal data 520, the reflected wave data 530, the number-of-reception channels table 540, transmission beam width data 570a, and the selection proportion conversion formula 560.

As the transmission beam width data 570a, the relationship between the angular range of the transmission beam width and the sensitivity illustrated in FIG. 11 and FIG. 12 is stored for each depth. Then, the allowable arrival angle range setter 410a refers to the transmission beam width data 570a, and sets the allowable arrival angle range corresponding to the allowable sensitivity level based on the relationship between the angular range of the transmission bam width corresponding to the depth of the processing target point and the sensitivity.

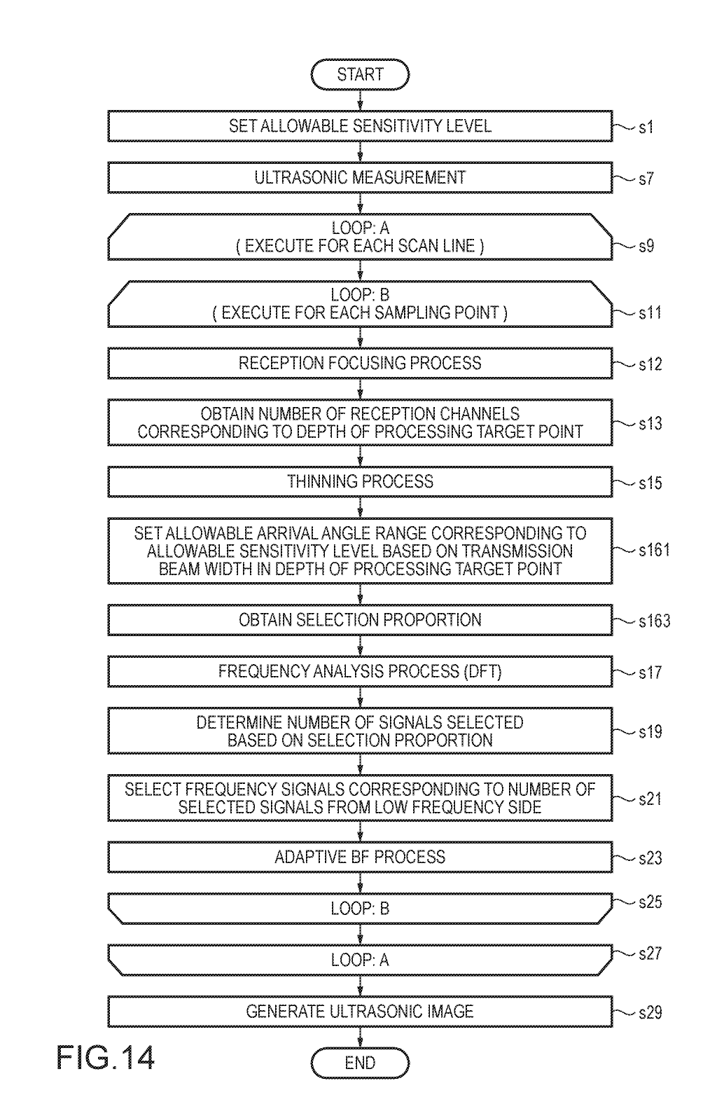

FIG. 14 is a flowchart showing a flow of a generation process of the ultrasonic image in the present modified example. It should be noted that in FIG. 14, substantially the same processing steps as in the embodiment described above are denoted by the same symbols. The present process can be realized by the arithmetic processor 370a retrieving the ultrasonic measurement program 510a from the storage 500a and then executing the ultrasonic measurement program 510a to thereby operate each section of the ultrasonic measurement device 10.

In the present modified example, the allowable sensitivity level is set in the step s1, and then the process proceeds to the step s7 to repeat the process on and after the ultrasonic measurement frame by frame. Then, after the thinning process in the step s15, the allowable arrival angle range setter 410a refers to the transmission beam width data 570a to read out the angle corresponding to the allowable sensitivity level set in the step s1 from the relationship between the angular range of the transmission beam width corresponding to the depth of the processing target point and the sensitivity, and then sets (step s161) the allowable arrival angle range. Then, the selection proportion calculator 420 obtains (step s163) the selection proportion corresponding to the angle obtained in the step s161 based on the selection proportion conversion formula 560. Subsequently, the process proceeds to the step s17.

According to the present modified example, by setting the allowable sensitivity level using the transmission beam width of the ultrasonic beam corresponding to the depth, it is possible to determine the number of frequency signals yₙ selected from the frequency signals yₖ, and thus substantially the same advantage as in the embodiment described above can be exerted.

### Modified Example 2

A harmonic mode is one of measurement modes of the ultrasonic measurement performed by the ultrasonic probe 16. The harmonic mode is a mode for performing the harmonic imaging process of extracting a harmonic component to generate the ultrasonic image. According to the harmonic imaging process, it is possible to image the harmonic component generated by the ultrasonic wave in the process of propagating through the living body, and thus it is possible to improve the resolution and the contrast. The embodiment described above can also be applied to the case of performing the ultrasonic measurement in the harmonic mode described above in substantially the same manner. Specifically, it is sufficient to prepare the number-of-reception channels table and the reception directional characteristics data, or the transmission beam width data based on the frequency of the harmonic component to be extracted.

### Modified Example 3

Further, although in the embodiment described above, the adaptive BF process is illustrated as the beam forming process, the invention can also be applied to the case of performing the non-adaptive beam forming process, in which the weighted addition is performed on the channel signals from the respective channels using a predetermine fixed addition weight, in substantially the same manner, and it is possible to obtain substantially the same advantage.

The entire disclosure of Japanese Patent Application No. 2016-187405 filed Sep. 26, 2016 is expressly incorporated by reference herein.

What is claimed is:

1. An ultrasonic measurement device comprising:
an ultrasonic probe having a plurality of ultrasonic elements arranged, each of the ultrasonic elements being adapted to transmit and receive an ultrasonic beam; and
an arithmetic processor adapted to perform a reduction process of reducing an amount of information of received signals received by the respective ultrasonic elements based on a reception frequency, then perform a beam forming process on the signals, on which the reduction process has been performed, to generate an ultrasonic image.

2. The ultrasonic measurement device according to claim 1, wherein
the arithmetic processor performs, as a part of the reduction process,
a frequency analysis process of performing frequency analysis on the received signals of the respective ultrasonic elements to transform the received signals into a plurality of frequency signals, and
a selection process of selecting signals with given frequency components from the frequency signals to thereby eliminate a signal other than the given frequency components.

3. The ultrasonic measurement device according to claim 2, wherein
the arithmetic processor performs
setting an allowable arrival angle range of a side lobe allowed to be received,
obtaining a selection proportion of the ultrasonic elements corresponding to the allowable arrival angle range, and
the selection process using components corresponding to the selection proportion on a low frequency side out of the reception frequencies obtained by the frequency analysis as the given frequency components.

4. The ultrasonic measurement device according to claim 3, wherein
the arithmetic processor sets an allowable level of the side lobe allowed to be received to thereby set the allowable arrival angle range fulfilling the allowable level based on reception directional characteristics related to the ultrasonic probe.

5. The ultrasonic measurement device according to claim 3, wherein
the arithmetic processor sets the allowable arrival angle range in accordance with depth of a processing target point of the beam forming process.

6. The ultrasonic measurement device according to claim 1, wherein
the arithmetic processor performs, as a part of the reduction process, a thinning process adapted to thin the received signals corresponding respectively to the ultrasonic elements in accordance with depth of a processing target point of the beam forming process.

7. The ultrasonic measurement device according to claim 6, wherein
the arithmetic processor performs the thinning process by thinning the received signals based on a pitch length of the ultrasonic elements corresponding to a propagatable frequency determined based on depth of the processing target point.

8. The ultrasonic measurement device according to claim 1, wherein
the arithmetic processor calculates weight based on the signals on which the reduction process has been performed, and performs the beam forming process as an adaptive beam forming process in which weighted addition is performed on the signals using the weight.

9. A method of controlling an ultrasonic measurement device adapted to perform ultrasonic measurement using an ultrasonic probe having a plurality of ultrasonic elements arranged, each of the ultrasonic elements being adapted to transmit and receive an ultrasonic beam, the method comprising:
performing a reduction process of reducing an amount of information of received signals received by the respective ultrasonic elements based on a reception frequency; and
generating an ultrasonic image by performing a beam forming process on the signals on which the reduction process has been performed.
